SEQUENCE LISTING (7 sequences)

SEQ ID NO: 1 - DNA, 83 bp, Homo sapiens
ccggagcccgacgccccgcgtccggacctggaggcagccctgggtctccggatcaggccagcggccaaagggtcgccgcacgc

SEQ ID NO: 2 - DNA, 83 bp, Artificial sequence
  Synthetic oligonucleotide
tcggagttcgacgtttcgcgttcggatttggaggtagttttgggttttcggattaggttagcggttaaagggtcgtcgtacgt

SEQ ID NO: 3 - DNA, 83 bp, Artificial sequence
  Synthetic oligonucleotide
ttggagtttgatgttttgtgtttggatttggaggtagttttgggtttttggattaggttagtggttaaagggttgttgtatgt

SEQ ID NO: 4 - DNA, 24 bp, Artificial sequence
  Synthetic oligonucleotide
gggaagtgttgtagggaggtattt

SEQ ID NO: 5 - DNA, 25 bp, Artificial sequence
  Synthetic oligonucleotide
aaaaccataatataaaaaccctaaa

SEQ ID NO: 6 - DNA, 34 bp, Artificial sequence
  Synthetic oligonucleotide
aggaagagaggggaagtgttgtagggaggtattt

SEQ ID NO: 7 - DNA, 56 bp, Artificial sequence
  Synthetic oligonucleotide
cagtaatacgactcactatagggagaaggctaaaaccataatataaaaaccctaaa

(19) United States
(12) Patent Application Publication
(10) Pub. No.: US 2021/0285052 A1
(43) Pub. Date: Sep. 16, 2021

(54) METHOD FOR DETECTING ABERRANT DNA METHYLATION OF THE TERT PROMOTER

(71) Applicant: ST. JUDE CHILDREN'S RESEARCH HOSPITAL, Memphis, TN (US)

(72) Inventors: Armita Bahrami, Memphis, TN (US); Seung Jae Lee, Collierville, TN (US)

(21) Appl. No.: 16/330,444

(22) PCT Filed: Sep. 12, 2017
(86) PCT No.: PCT/US2017/051099

§ 371 Date: Mar. 5, 2019

Related U.S. Application Data
(60) Provisional application No. 62/393,843, filed on Sep. 13, 2016.

(51) Int. Cl. C12Q 1/6886

(52) U.S. Cl. CPC C12Q 1/6886; C12Q 2600/106; C12Q 2600/154

(57) ABSTRACT

A method for detecting methylated CpG sites located in the promoter of the Telomerase Reverse Transcriptase (TERT) gene, using the restriction enzymes BsiWI and Hpy188I or Hpy99I, is provided. A kit for carrying out the method and methods for diagnosing cancer and assessing whether a mammal with cancer is a candidate for treatment with a demethylating agent is also provided.

### INTRODUCTION

This application claims the benefit of priority from U.S. Patent Application Ser. No. 62/393,843, filed Sep. 13, 2016, the content of which is incorporated herein by reference in its entirety.

This invention was made with government support under Grant Number P30CA021765 awarded by the National Institutes of Health. The government has certain rights in the invention.

### BACKGROUND

Telomeres are repetitive DNA structures at the ends of chromosomes that stabilize the genome by protecting chromosomes from degradation and end-to-end fusion. With each round of cell division, telomeres reduce in length. A cell with a critically shortened telomeres stops dividing enters a state of senescence. Cancer cells have the ability to maintain their telomere length and multiply indefinitely. The majority of malignant tumors (80% to 90%) overcome telomere shortening through aberrant upregulation of the enzyme telomerase, whose rate-limiting subunit is encoded by the Telomerase Reverse Transcriptase (TERT) gene. Telomerase activity and TERT mRNA levels are undetectable in untransformed somatic cells but are upregulated in the majority of cancer cells.

TERT expression is known to be activated in the setting of cancer by genetic alterations including promoter point mutations, or structural rearrangements, and whole gene amplification. Aberrant epigenetic changes of the TERT promoter, such as CpG dinucleotide methylation and activating histone modifications, have also been noted in cancer.

The specific genetic and epigenetic changes associated with TERT expression in each type of cancer is an emerging area of investigation. DNA methylation of a region of the TERT promoter −483 bp to −541 bp upstream of the translation start site (chr5: 1295587-1295645 [GRCh37/hg19]) has been associated with TERT expression in several types of cancer. Importantly, this region is observed to be unmethylated or less methylated in normal and benign neoplastic counterparts. The differential methylation pattern of the TERT promoter and its potential diagnostic value in differentiating between proliferative nodules in giant congenital nevi and malignant melanoma associated with these nevi have been previously demonstrated (Fan, et al. (2015) J. Invest. Dermatol. 136:339-42). Indeed, the use of promoter methylation as a biomarker of cancer is expected to become increasingly relevant in the future.

A major barrier in integrating this knowledge discovery into clinical practice is the technical difficulty in methylation profiling of the promoter sequence. Aberrant methylation of the TERT promoter can be assayed by sophisticated techniques, such as SEQUENOM® and pyrosequencing (Castelo-Branco, et al. (2013) Lancet Oncol. 14:534-42; WO 2013/173912), methylation-sensitive single nucleotide primer extension (U.S. Pat. No. 6,251,594), or next-generation bisulfite sequencing (Fan, et al. ((2015) J. Invest. Dermatol. 136:339-42).

However, many of these assays are expensive and can only be performed on a large batch of samples. Therefore, they cannot be easily transferred to the diagnostic platform in clinical laboratories. Accordingly, there is a need in the art for a simple, reliable, specific, and accurate technique that is easily transferrable to clinical use.

### SUMMARY OF THE INVENTION

This invention provides a method for detecting methylated CpG sites in the promoter of the TERT gene by (a) obtaining at least a portion of the TERT promoter from a sample (e.g., −483 bp to −541 bp upstream of the translation start site of TERT), (b) digesting the TERT promoter with (i) BsiWI and (ii) Hpy188I or Hpy99I, and (c) determining the presence of restricted fragments of the TERT promoter. In some embodiments, step (a) includes (i) obtaining nucleic acids from a tissue sample (e.g., tissue having or suspected of having cancer cells), and (ii) subjecting the nucleic acids to bisulfite conversion. A kit containing (a) primers for obtaining at least a portion of the TERT promoter; and (b) BsiWI, Hpy188I, and/or Hpy99I is also provided. Methods for diagnosing cancer and assessing whether a mammal with cancer is a candidate for treatment with a demethylating agent are also included within the scope of this invention.

### BRIEF DESCRIPTION OF THE DRAWINGS

FIG. 1 shows fragments resulting from the digestion of bisulfite amplicons of the commercially available 0% and 100% methylated TERT promoter with BsiWI and Hpy188I.

FIG. 2 shows the 26 CpG sites residing in the TERT promoter as well as the small-sized fragments resulting from digestion of the 285 bp bisulfite amplicon generated using PCR primer pair 2 (Table 1).

FIG. 3 shows the nucleic acid sequences of the TERT promoter (−483 bp to −541 bp upstream of the translation start site; SEQ ID NO:1) as well as the location of restriction sites for the BsiWI and the Hpy188I enzymes in the CpG-methylated (SEQ ID NO:2) compared to the CpG-unmethylated (SEQ ID NO:3) TERT promoter after bisulfite conversion.

FIG. 4 shows the DNA fragments (arrows) resulting from digestion of the methylated TERT promoter from malignant melanoma samples arising in giant congenital nevi (S2, S1, S1001, S21) digested with BsiWI and the Hpy188I as compared to unmethylated TERT promoter from benign proliferative nodules in giant congenital nevi (S26 and S11).

### DETAILED DESCRIPTION OF THE INVENTION

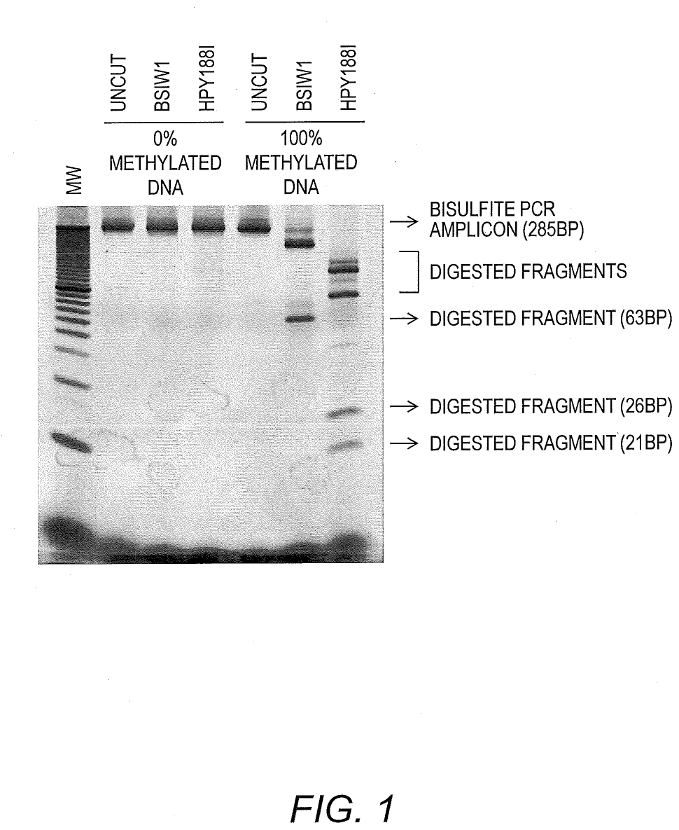

Aberrant CpG methylation within a particular region of the TERT promoter is linked to cancer. This invention provides a method for detecting these methylated CpG sites within this particular region of the TERT promoter (−483 bp to −541 bp upstream of the translation start site) by using the restriction enzymes BsiWI and Hpy188I or Hpy99I. Digestion with these enzymes yields small DNA fragments of specific sizes that are detectable by gel electrophoresis, which indicate the methylation status of the CpG sites of interest (FIG. 1). The assay can discriminate between benign/low-grade (CpG-unmethylated TERT promoter) and overtly malignant (CpG-methylated TERT promoter) melanomas, and has also been used to characterize tumors of other histologic categories. Therefore, the method of this invention is of use in the diagnosis of several types of cancer as well as the identification of tumors that might be responsive to demethylating agents.

The methods of this invention involve the steps of obtaining at least a portion of the TERT promoter from a sample; digesting the TERT promoter with restriction enzyme (i) BsiWI and (ii) Hpy188I or Hpy99I; and determining the presence of restricted fragments of the TERT promoter. As is known in the art, “TERT” refers to mammalian telomerase reverse transcriptase and includes human (hTERT) and non-human TERT. The term “TERT promoter” refers to nucleic acids encompassing the region upstream of the translation start site of the gene encoding TERT, i.e., the TERT gene, namely, the region upstream of the translation start site beginning at position −1 and extending upstream therefrom, in particular nucleotides in the proximal region of the promoter spanning position −1 to approximately −1000.

The TERT promoter can be obtained from any biological sample for which the methylation status of the TERT promoter is to be determined. However, in particular embodiments, the TERT promoter is obtained from a tissue sample having or suspected of having cancer cells. The term “cancer” is used herein to encompass cancers (malignant neoplasms) including, but not limited to, carcinoma such as bladder, breast, colon, small bowel, kidney, brain, liver, lung (including small cell lung cancer), esophagus, gallbladder, ovary, uterus, pancreas, stomach, cervix, thyroid, parathyroid, prostate, adrenal gland, liver, oral cavity, larynx, mucosa, and skin; mesenchymal tumors including sarcomas such as osteosarcoma, synovial sarcoma, rhabdomycosarcoma, and Kaposi sarcoma; hematopoietic tumors of lymphoid lineage, including leukemia, acute lymphocytic leukemia, acute lymphoblastic leukemia, B-cell lymphoma, T-cell lymphoma, Hodgkin lymphoma, non-Hodgkin lymphoma, hairy cell lymphoma, and Burkitt lymphoma; hematopoietic tumors of myeloid lineage, including acute and chronic myelogenous leukemias, myelodysplasia syndrome and promyelocytic leukemia; tumors of mesenchymal origin such as gastrointestinal stromal tumors, sex cord gonadal stromal tumors, and endometrial stromal sarcoma; tumors of the central and peripheral nervous system such as astrocytoma, neuroblastoma, glioma, and malignant peripheral nerve sheath tumors; and other tumors, including melanoma, germ cell tumors such as seminoma, myoepithelial tumors, and pediatric cancers of embryonal and other origins such as Ewing sarcoma/PNET, Wilms tumors and hepatoblastoma.

Tissue samples include patient-derived primary biological samples including, for example, blood, serum, plasma, and urine, or biopsied tissue including, but not limited to, tumor tissue, pleural effusions obtained from a mammal, or formalin-fixed paraffin-embedded samples thereof. The sample can be obtained using techniques well established and known to those of skill in the art, and will vary with the sample type, as one of skill in the art will appreciate. Different techniques that can be used to obtain a tumor sample include standard biopsy, needle biopsy, endoscopic biopsy, bone marrow biopsy, and combination techniques that employ biopsy and imaging. Needle biopsy is commonly used to obtain a sample from a tumor under the skin surface (e.g., breast tumor). A sample of a brain tumor can be obtained by standard biopsy, stereotactic biopsy, or neuroendoscopy. In general, a suitable tumor sample needs to contain up to approximately ng to 1 μg of nucleic acid for methylation to be determined within the target region of the TERT promoter.

Biological samples can be obtained from a mammal suspected of or exhibiting one or more signs or symptoms of cancer. The term “mammal” is used herein to encompass organisms in which telomerase is not ubiquitously expressed. Such mammals include, for example, humans, cats, dogs, horses, cattle, pigs, and various wild animals. Mice and other mammals in which telomerase is ubiquitously expressed are not included within this definition.

Nucleic acids encompassing the TERT promoter can be isolated from a sample by conventional methods that preserve the methylation status of a nucleic acid molecule, including, e.g., chemical extraction techniques using phenol-chloroform, guanidine-containing solutions, or CTAB-containing buffers. For convenience, commercial DNA extraction kits such as the QIAAMP DNA Blood Minikit from QIAGEN (Chatsworth, Calif.) or the Extract-N-Amp Blood Kit from Sigma-Aldrich (St. Louis, Mo.) can be used.

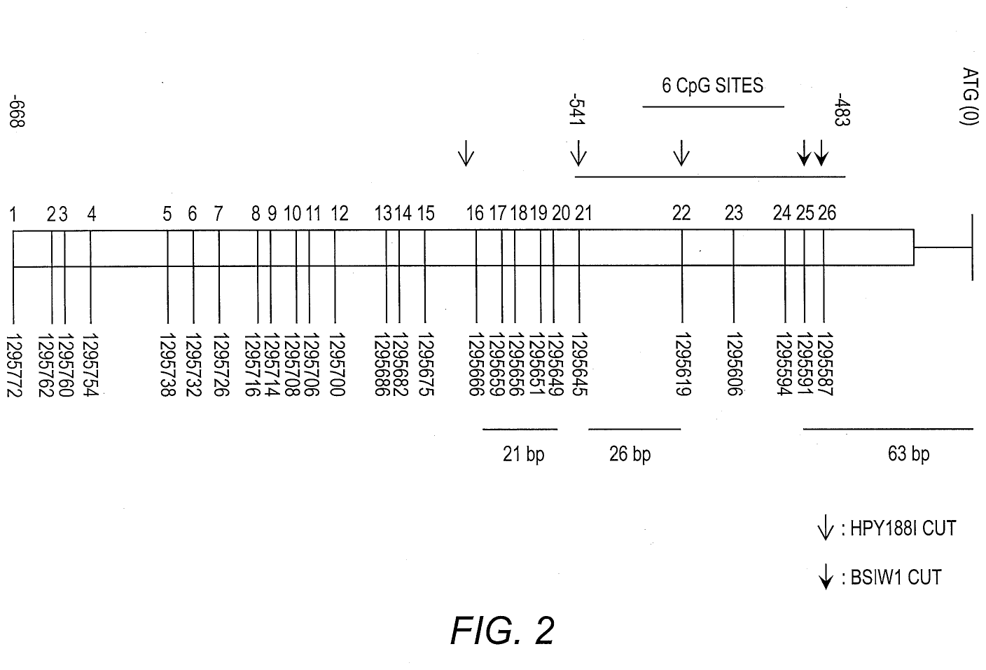

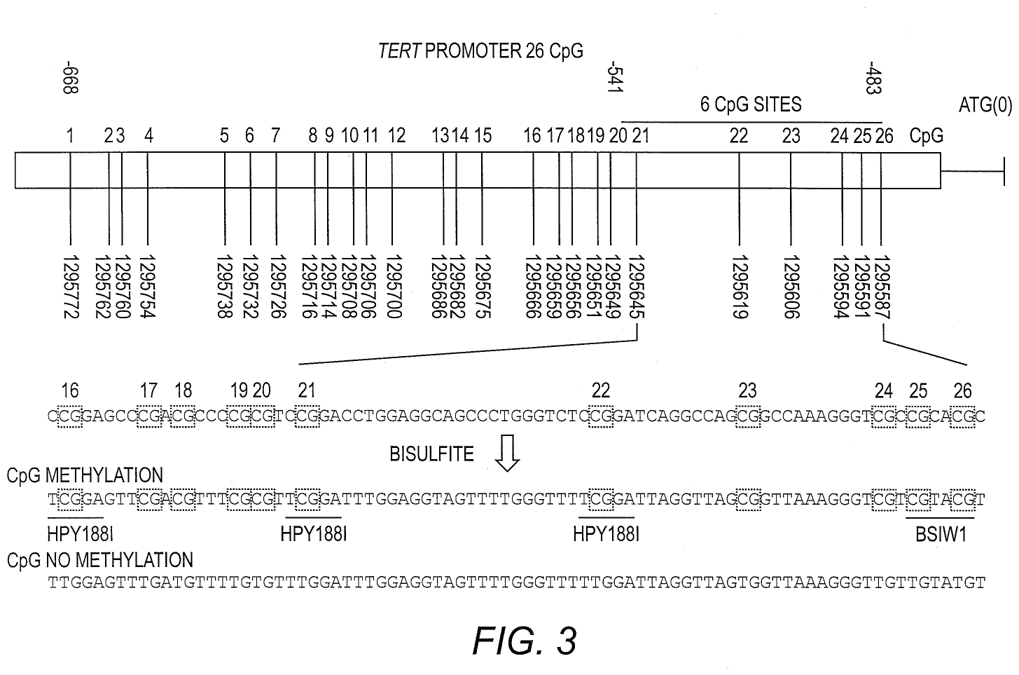

After obtaining an appropriate nucleic acid sample, the methylation status of a target region within the TERT promoter is determined. In particular, methylation of the CpG sites located −483 bp to −541 bp upstream of the translation start site, i.e., nucleotides 1295587 to 1295645 on chromosome 5 of the human genome 19^{th} assembly, is determined. More specifically, methylation at CpG sites at positions 1295587, 1295591, 1295594, 1295619, 1295645, 1295656, 1295659, and/or 1295666 is determined (see FIG. 2 and FIG. 3). This can include the presence or absence of methylation at a particular CpG site or degree of methylation of the TERT promoter at −483 bp to −541 bp. The term “degree of methylation” is used herein to refer to the extent to which the TERT promoter at −483 bp to −541 bp is methylated, i.e., whether one, two, three, four, five, or six CpG sites of interest are methylated.

To determine the presence, absence or degree of methylation at CpG sites within the TERT promoter, the target region is amplified using well established methods such as PCR. In particular, the nucleic acid sample is bisulfite-converted using bisulfite PCR. Bisulfite conversion involves the replacement of unmethylated cytosines with uracil. Unlike normal PCR, bisulfite PCR primers are typically long (usually 26 to 30 bases) and the amplicon size is relatively short (150 to 300 bp). Ideally, the primers do not contain CpG sites. Usually, 35 to 40 cycles are required for successful amplification, with annealing temperatures of 55° C. to 60° C. The use of hot-start polymerases for non-specific amplification is relatively common with bisulfite-converted DNA because it is AT-rich. Bisulfite conversion is conventional in the art and can be carried out using commercially available reagents or kits.

By way of illustration, the bisulfite conversion reaction can be carried out by denaturing approximately 1 μg of genomic DNA (amount of DNA can be lower when using micro-dissected DNA specimens) for 15 minutes at 45° C. with N NaOH, followed by incubation with 0.1 M hydroquinone and 3.6 M sodium bisulfite (pH 5.0) at 55° C. for 12 hours (appropriate range is 4 to 12 hours). The DNA is then purified from the reaction mixture using standard (commercially available) DNA miniprep columns or other standard techniques. The purified DNA sample is resuspended in 55 μL of water, and 5 μL of 3 N NaOH is added for a desulfonation reaction, preferably performed at 40° C. for 5 to 10 minutes. The DNA sample is then precipitated with ethanol and washed before being resuspended in an appropriate volume of water.

Bisulfite PCR amplification can be performed by standard PCR techniques, following the manufacturer's instructions. For example, approximately 1 to 2 μL of the bisulfite-treated DNA is used as a template for strand-specific PCR amplification in a region of interest. A typical PCR reaction includes an initial denaturation of 94° C. for 3 minutes followed by a cycle of 94° C. of 30 seconds, 68° C. for 30 seconds, and 72° C. for 30 seconds for a total of 30 cycles. The PCR reactions can be performed in a volume of 25 μL under conditions of approximately 50 ng bisulfite-converted DNA (lower for micro-dissected samples), 10 mM Tris-HCl (pH 8.3), 1.5 mM MgCl₂, 50 mM KCl, 0.1% gelatin/mL, 100 μM of each of dNTP, 0.5 μM final concentration of each primer, and 1 unit of TAQ polymerase. In certain embodiments, the TERT promoter is amplified by using the primer pairs provided in Table 1.

TABLE 1

| Primer | | Sequence | SEQ ID |
|---|---|---|---|
| Pair | Primer | (5′→3′) | NO: |
| 1 | Forward | GGGAAGTGTTGTAGGGAGGTATTT | 4 |
| | Reverse | AAAACCATAATATAAAAACCCTAAA | 5 |
| 2 | Forward | AGGAAGAGAGGGGAAGTGTTGTAGGGAGGTATTT | 6 |
| | Reverse | CAGTAATACGACTCACTATAGG | 7 |
| | | GAGAAGGCTAAAACCATAATATAAAAACCCTAAA | |

The sequence amplified between the primers listed in Table 1 is located between position 1295553 and 1295796 of Human Genome 19 assembly—chr5 (GRCh37/hg19). See FIG. 2 and FIG. 3.

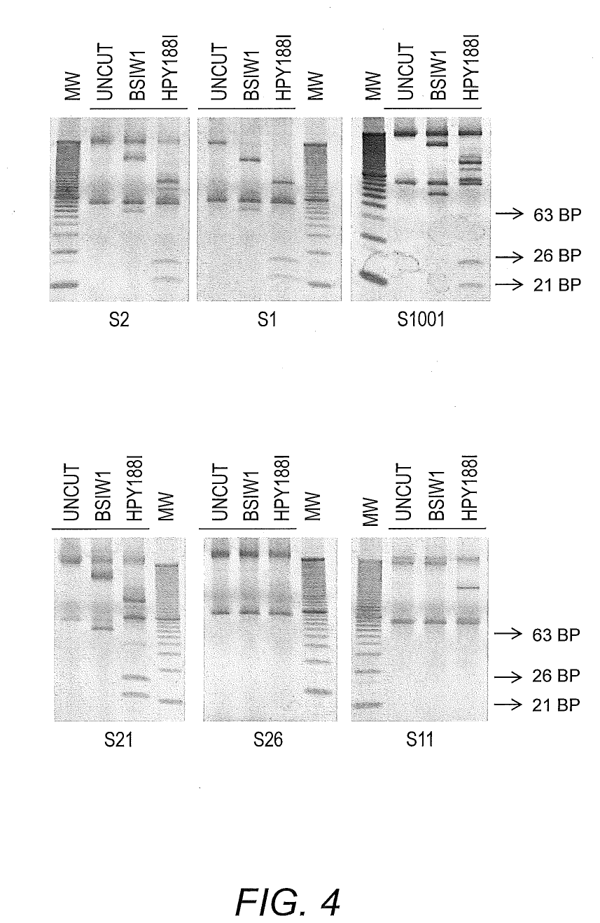

Since the correlation of methylation upstream of the TERT transcription start site and tumor severity may vary across different types of cancer, tissue- and tumor-specific controls are important for reliable interpretation. After the TERT promoter is bisulfite-modified and amplified by PCR, the amplicon is independently digested with restriction enzymes BsiWI and Hpy188I or Hpy99I. BsiWI recognizes the restriction site 5′-C^{↓}GTACG-3′ and cuts the methylated CpG sites at positions 1295587 and 1295591 of the TERT promoter (see FIG. 2 and FIG. 3). Hpy188I recognizes the restriction site 5′-TCN^{↓}GA-3′ and cuts the methylated CpG sites at positions 1295619, 1295645, and 1295666 (see FIG. 2 and FIG. 3). Hpy99I recognizes the restriction site 5′-CGWCG^{↓}-3′ and cuts the methylated CpG sites at positions 1295591 and 1295594 or 1295656 and 1295659. If two or more of the CpG sites located at positions 1295587, 1295591, 1295594, 1295619, and 1295645 among six CpG sites of the TERT promoter are methylated, the cytosines at these positions are retained, unique restriction sites are formed, and the restriction enzymes digest the amplicon into smaller fragments (see FIG. 1 and FIG. 4). If the CpG sites located at positions 1295587, 1295591, 1295594, 1295619, and 1295645 of the TERT promoter are not methylated, the cytosines therein get converted to uracils and the restriction enzyme recognition sequences are not generated such that the enzymes cannot cut the amplicon (see FIG. 1). To determine whether the restriction enzyme(s) digest the TERT promoter, the nucleic acid sample are subjected to size separation by slab or capillary gel electrophoresis, using known protocols. BsiWI digests the amplicons that are methylated at both the CpG sites 1295587 and 1295591 into small DNA fragments of specific sizes. For example, when a nucleic acid amplified with the primer pair 2 (Table 1) is subjected to digestion with the BsiWI enzyme, a small 63 bp restriction fragment is created if the sequence is methylated at both the CpG sites 1295587 and 1295591 (see FIG. 2 and FIG. 4). By comparison, subjecting the sample amplified molecule to digestion with Hpy188I generates small-sized fragments, 21 bp and 26 bp, if the amplicon is methylated at the CpG sites 1295619, 1295645 and 1295666 (see FIG. 2 and FIG. 4). In amplicons with only one single methylated CpG site, the restriction enzymes do not generate the small DNA fragments of specific sizes. Consequently, methylation in at least two CpG sites is needed for the enzymes to generate the specific sized fragments. The presence of digested fragments with both BsiWI and Hpy188I indicates that the sample is aberrantly methylated at the CpG sites of interest in the TERT promoter.

For comparative purposes, methylation of CpG sites within the same target region of the TERT promoter can be similarly determined using an appropriate technique in a corresponding control or normal biological sample. A control or normal biological sample is a noncancerous sample of a corresponding biological sample from the same mammal or from a mammal this is determined to be comparable, e.g., in the case of a test sample from a human, a control sample can be obtained from another human, who may or may not be of the same age or sex. Generally, a median value of methylation determined within the target TERT promoter region from multiple control samples aids in providing an accurate control value. Note that normal biological samples exhibit a small degree or baseline level of methylation that can vary by sample type. Thus, determining the degree of methylation in a TERT promoter region of a control sample helps in the accurate analysis of the actual degree of methylation in a test sample, i.e., whether hypermethylation actually exists in the test sample.

The detection of methylated CpG sites in the TERT promoter finds application in the diagnosis of cancer as well as assessing the response of a tumor to targeted therapy with a demethylating agent. Accordingly, this invention also provides a method for diagnosing cancer by determining the presence of methylated CpG sites in the TERT promoter from a sample. This method involves obtaining at least a portion of the TERT promoter from a sample; digesting the TERT promoter with restriction enzyme BsiWI and Hpy188I or Hpy99I; and determining the presence of restricted fragments of the TERT promoter. The presence of restricted fragments of the TERT promoter indicates methylation in the target region of the TERT promoter and the presence of cancer in the mammal. In particular embodiments, this method is used to discriminate between benign/low-grade (CpG-unmethylated TERT promoter) and overtly malignant tumors (CpG-methylated TERT promoter). For comparative purposes, the method can also include the use of a control as described herein.

This invention also provides a method for assessing whether a mammal with cancer is a candidate for treatment with a demethylating agent. This method involves the steps of obtaining at least a portion of the TERT promoter from a sample; digesting the TERT promoter with restriction enzyme BsiWI and Hpy188I or Hpy99I; and determining the presence of restricted fragments of the TERT promoter. For comparative purposes, this method can also include the use of a control as described herein. The analysis of results obtained will help determine whether the mammal is a candidate for treatment with one or more demethylating agents. When the results indicate that the target region of the TERT promoter is methylated or hypermethylated, then the mammal is a likely candidate for treatment with a demethylating agent. On the other hand, if there is no methylation or hypermethylation of the target region of the TERT promoter, then the mammal is likely not a candidate for treatment with a demethylating agent. Demethylating agents include, but are not limited to, 5 azacytidine (VIDAZA) and derivatives thereof, such as 5-aza-2′-deoxycytidine (DECITABINE).

What is claimed is:

1. A method for detecting methylated CpG sites in the promoter of the Telomerase Reverse Transcriptase (TERT) gene comprising
(a) obtaining at least a portion of the TERT promoter from a sample,
(b) digesting the TERT promoter with (i) BsiWI and (ii) Hpy188I or Hpy99I, and
(c) determining the presence of restricted fragments of the TERT promoter, thereby detecting methylated CpG sites in the promoter of the TERT.

2. The method of claim 1, wherein step (a) comprises
(i) obtaining nucleic acids from a tissue sample, and
(ii) subjecting the nucleic acids to bisulfite conversion.

3. The method of claim 1, wherein the portion of the TERT promoter comprises −483 bp to −541 bp upstream of the translation start site of TERT.

4. The method of claim 1, wherein the sample comprises tissue having or suspected of having cancer cells.

5. A kit comprising
(a) primers for obtaining at least a portion of the TERT promoter; and
(b) BsiWI and Hpy188I or Hpy99I.

6. A method for diagnosing cancer comprising
(a) obtaining at least a portion of the TERT promoter from a sample from a subject having or suspected of having cancer,
(b) digesting the TERT promoter with (i) BsiWI and (ii) Hpy188I or Hpy99I, and
(c) determining the presence of restricted fragments of the TERT promoter,
wherein the presence of restricted fragments of the TERT promoter indicates that the TERT promoter is methylated and the subject has cancer.

7. A method for assessing whether a mammal with cancer is a candidate for treatment with a demethylating agent comprising
(a) obtaining at least a portion of the TERT promoter from a sample from a subject having or suspected of having cancer,
(b) digesting the TERT promoter with (i) BsiWI and (ii) Hpy188I or Hpy99I, and
(c) determining the presence of restricted fragments of the TERT promoter,
wherein the presence of restricted fragments of the TERT promoter indicates that the TERT promoter is methylated and the subject is a candidate for treatment with a demethylating agent.
